(19) United States
(12) Patent Application Publication
(10) Pub. No.: US 2026/0139224 A1
(43) Pub. Date: May 21, 2026

(54) REAGENTS, REAGENT KITS, AND CULTURE MEDIA FOR ACTIVATING AND EXPANDING IMMUNE CELLS AND USES THEREOF

(71) Applicant: Nanotein Technologies, Inc., Berkeley, CA (US)

(72) Inventors: Curtis Daniel HODGE, Pinole, CA (US); Patrick Michael CARNEY, Berkeley, CA (US); Zachary Ibrahim IMAM, Concord, CA (US)

(73) Assignee: Nanotein Technologies, Inc., Berkeley, CA (US)

(21) Appl. No.: 19/301,774

(22) Filed: Aug. 15, 2025

Related U.S. Application Data
(63) Continuation-in-part of application No. 18/731,209, filed on May 31, 2024, now Pat. No. 12,473,529.
(60) Provisional application No. 63/514,243, filed on Jul. 18, 2023.

(51) Int. Cl. C12N 5/0783

(52) U.S. Cl. CPC C12N 5/0636; C12N 2501/51; C12N 2501/515; C12N 2501/90; C12N 2501/999

(57) ABSTRACT

Disclosed herein are reagents, reagent kits, and culture media for activating and expanding immune cells in vitro. In some embodiments, the reagents, reagent kits, and culture media comprise a Quillaja saponin. In other embodiments, the reagents, reagent kits, and culture media comprise a squalene-based emulsion. Also disclosed are methods for activating and expanding immune cells in vitro using the reagents, reagent kits, and culture media.

### CROSS-REFERENCE TO RELATED APPLICATION

This application is a continuation-in-part of U.S. patent application Ser. No. 18/731,209 filed on May 31, 2024, which claims the benefit of priority to U.S. Patent Application No. 63/514,243 filed on Jul. 18, 2023, the content of which is incorporated herein by reference in its entirety.

### REFERENCE TO AN ELECTRONIC SEQUENCE LISTING

This application contains a Sequence Listing which has been submitted via Patent Center and is hereby incorporated by reference in its entirety. Said .xml copy, created on Aug. 15, 2025 is named 04_NNTNNZ00201_sequence_listing.xml and is 5,183 bytes in size.

### TECHNICAL FIELD

This disclosure relates generally to the field of cell therapies and, more specifically, to reagents, reagent kits, and culture media for activation and expansion of immune cells and uses thereof.

### BACKGROUND

Adjuvants are substances that are used to increase the efficacy or potency of a drug or vaccine. The use of adjuvants began with aluminum salts after it was found that they strengthened the body's immune response to diphtheria and tetanus vaccines [1]. Modern day adjuvants include, for example, oil-in-water emulsions and saponin-based adjuvants extracted from the Chilean soapbark tree. Oil-in-water emulsions are a commonly used type of vaccine adjuvant that includes AS03, a squalene-based emulsion, which has been used as an adjuvant for influenza A (H5N1) [2].

Saponins come from the bark, stem, roots, and flowers of several plant species and are made up of sterol glycosides and triterpenoid glycosides [3] Quil A is a saponin-based adjuvant used in a wide variety of veterinary vaccines [4]. QS-21 is a purified fraction of the Quillaja saponin composed of two isomeric bidesmosidic saponins from the Quillaja saponaria Molina tree [5]. QS-21 is currently used in vaccines for malaria and shingles [6].

While adjuvants have been used safely in vaccines for decades, the active ingredients of such adjuvants have, heretofore, not been explored in the biomanufacturing of cell-based immunotherapies in vitro. More specifically, squalene-based emulsions and saponins have not been explored for activating and expanding immune cells in vitro as part of a biomanufacturing process for cell-based immunotherapies.

### SUMMARY

Disclosed are reagents or additives, reagent kits, and culture media capable of facilitating the activation and expansion of immune cells.

In some embodiments, disclosed is a reagent for activating and expanding immune cells in vitro comprising a Quillaja saponin in solution.

In some embodiments, the Quillaja saponin can be derived from the inner bark or cortex of the Quillaja saponaria Molina tree.

In some embodiments, the Quillaja saponin can be Quil A.

In some embodiments, the Quillaja saponin can be a purified Quillaja saponin.

In certain embodiments, the purified Quillaja saponin can be QS-21.

In some embodiments, the reagent can comprise QS-21. In these embodiments, the reagent comprising the QS-21 can be added to a T cell culture medium to cause the QS-21 concentration to be between about 1.5 μg/mL and about 3.9 μg/mL (where the concentrations refer to μg of QS-21 per mL of culture medium). The reagent can also comprise an anti-CD3 and anti-CD28 T-cell activation reagent.

The anti-CD3 and anti-CD28 T-cell activation reagent can comprise a plurality of self-assembling protein nanoparticles decorated with anti-CD3 antibodies and anti-CD28 antibodies. At least one of the self-assembling protein nanoparticles can comprise a protein cage polypeptide assembled into a three-dimensional macromolecular structure and can serve as a scaffold for the anti-CD3 antibodies and the anti-CD28 antibodies. In some embodiments, the three-dimensional macromolecular structure can be a tetrahedral pyramid.

In other embodiments, the reagent can comprise Quil A. In these embodiments, the reagent comprising the Quil A can be added to a T cell culture medium to cause the Quil A concentration to be between about 8 μg/mL and 18 μg/mL (where μg/mL is μg of the Quil A per mL of the culture medium). The reagent can also comprise the anti-CD3 and anti-CD28 T-cell activation reagent.

In some embodiments, the immune cells can be human donor peripheral blood immune cells.

In some embodiments, the immune cells can be live T cells.

In some embodiments, the live T cells can be CD4⁺ T cells and CD8⁺ T cells.

Also disclosed is a culture medium for activating and expanding immune cells in vitro comprising a Quillaja saponin.

In some embodiments, the Quillaja saponin can be derived from the inner bark or cortex of the Quillaja saponaria Molina tree.

In some embodiments, the culture medium can be a serum-free culture medium. In other embodiments, the culture medium can comprise serum.

In some embodiments, the culture medium can be a T-cell expansion medium.

In some embodiments, the Quillaja saponin can be pre-mixed into the culture medium.

In some embodiments, the Quillaja saponin can be Quil A.

In some embodiments, the Quillaja saponin can be a purified Quillaja saponin.

In some embodiments, the purified Quillaja saponin can be QS-21.

In some embodiments, the culture medium can comprise between about 1.5 μg/mL and 3.9 μg/mL of QS-21.

In other embodiments, the culture medium can comprise between about 8 μg/mL and 18 μg/mL of QS-21.

In additional embodiments, the culture medium can comprise between about 8 μg/mL and 18 μg/mL of Quil A.

In some embodiments, the immune cells can be human donor peripheral blood immune cells.

In some embodiments, the immune cells can be live T cells.

In some embodiments, the live T cells can be CD4⁺ T cells.

Also disclosed is a method of activating and expanding immune cells in vitro. The method can comprise adding a reagent comprising a Quillaja saponin to a culture medium comprising the immune cells (e.g., CD4⁺ T cells and CD8⁺ T cells). The reagent can comprise the anti-CD3 and anti-CD28 T-cell activation reagent.

In some embodiments, the Quillaja saponin can be Quil A.

In some embodiments, the Quillaja saponin can be a purified Quillaja saponin.

In some embodiments, the purified Quillaja saponin can be QS-21.

In some embodiments, the reagent can comprise QS-21. In these embodiments, the reagent comprising the QS-21 can be added to the culture medium (e.g., a serum-free T cell culture medium or a T cell culture medium comprising serum) to cause the QS-21 concentration to be between about 1.5 μg/mL and about 3.9 μg/mL (where the concentrations refer to μg of QS-21 per mL of culture medium).

In alternative embodiments, the reagent comprising the QS-21 can be added to the culture medium (e.g., a serum-free T cell culture medium or a T cell culture medium comprising serum) to cause the QS-21 concentration to be between about 8 μg/mL and about 18 μg/mL (where the concentrations refer to μg of QS-21 per mL of culture medium).

In other embodiments, the reagent can comprise Quil A. In these embodiments, the reagent comprising the Quil A can be added to the culture medium to cause the Quil A concentration to be between about 8 μg/mL and 18 μg/mL (where μg/mL is μg of the Quil A per mL of the culture medium).

Also disclosed is another method of activating and expanding immune cells in vitro by first activating a population of immune cells with an anti-CD3 and anti-CD28 T-cell activation reagent prior to adding the Quillaja saponin.

The anti-CD3 and anti-CD28 T-cell activation reagent can comprise a plurality of self-assembling protein nanoparticles decorated with anti-CD3 antibodies and anti-CD28 antibodies. At least one of the self-assembling protein nanoparticles can comprise a protein cage polypeptide assembled into a three-dimensional macromolecular structure and can serve as a scaffold for the anti-CD3 antibodies and the anti-CD28 antibodies. In some embodiments, the three-dimensional macromolecular structure can be a tetrahedral pyramid.

In some embodiments, the Quillaja saponin can be dissolved in water.

In some embodiments, the Quillaja saponin can be initially in a lyophilized form before being dissolved in water.

In some embodiments, the Quillaja saponin can be derived from the inner bark or cortex of the Quillaja saponaria Molina tree.

In some embodiments, the Quillaja saponin can be Quil A. In certain embodiments, the concentration of Quil A added to a culture medium comprising the immune cells can be between about 8 μg/mL and 18 μg/mL (where μg/mL is μg of the Quillaja saponin per mL of the culture medium).

In some embodiments, the Quillaja saponin can be a purified Quillaja saponin such as QS-21. In certain embodiments, the concentration of QS-21 added to a culture medium comprising the immune cells can be between about 1.5 μg/mL and 3.9 μg/mL (where μg/mL is μg of the Quillaja saponin per mL of the culture medium). In other embodiments, the concentration of QS-21 added to a culture medium comprising the immune cells can be between about 8 μg/mL and 18 μg/mL (where μg/mL is μg of the Quillaja saponin per mL of the culture medium).

In some embodiments, the method can further comprise dissolving the QS-21 in phosphate buffered saline (PBS) prior to adding the QS-21 to a culture medium comprising the population of immune cells. The QS-21 can be initially in a lyophilized form.

In some embodiments, the immune cells can be human donor peripheral blood immune cells.

In some embodiments, the immune cells can be live T cells.

In some embodiments, the live T cells can be CD4⁺ T cells and CD8⁺ T cells.

In some embodiments, disclosed is an aqueous solution for activating and expanding immune cells in vitro comprising DL-α-tocopherol, squalene oil, and a nonionic surfactant.

In some embodiments, the DL-α-tocopherol can be present in an amount from 3% to 7% (v/v). The squalene oil can be present in an amount from 3% to 7% (v/v). The nonionic surfactant can be present in an amount from 1% to 2.5% (v/v).

In some embodiments, the DL-α-tocopherol can be present in an amount from 0.50% to 4.0% (w/v). The squalene oil can be present in an amount from 0.50% to 4.0% (w/v). The nonionic surfactant can be present in an amount from 0.20% to 2.0% (w/v).

In some embodiments, the nonionic surfactant can be polysorbate 80.

In some embodiments, the immune cells can be human donor peripheral blood immune cells.

In some embodiments, the immune cells can be live T cells.

In some embodiments, the live T cells can be CD4⁺ T cells.

In some embodiments, the live T cells can be CD8⁺ T cells.

In some embodiments, disclosed is a culture medium for activating and expanding immune cells in vitro comprising a squalene-based emulsion. The squalene-based emulsion can comprise DL-α-tocopherol, squalene oil, and a nonionic surfactant.

In some embodiments, the culture medium can be a serum-free culture medium. In other embodiments, the culture medium can comprise serum.

In some embodiments, the culture medium can be a T-cell expansion medium.

In some embodiments, the squalene-based emulsion can be pre-mixed into the culture medium.

In some embodiments, the immune cells can be human donor peripheral blood immune cells.

In some embodiments, the immune cells can be live T cells.

In some embodiments, the live T cells can be CD4⁺ T cells.

In some embodiments, the live T cells can be CD8⁺ T cells.

In some embodiments, disclosed is a method of activating and expanding immune cells in vitro comprising adding a reagent to a culture medium comprising the immune cells. The reagent can comprise an aqueous solution comprising DL-α-tocopherol, squalene oil, and a nonionic surfactant. In other embodiments, the reagent can comprise the anti-CD3 and an anti-CD28 activation reagent.

Also disclosed is another method of activating and expanding immune cells in vitro.

The method can comprise activating the population of immune cells with an anti-CD3 and an anti-CD28 activation reagent prior to adding an aqueous solution comprising DL-α-tocopherol, squalene oil, and a nonionic surfactant.

In some embodiments, the DL-α-tocopherol can be present in an amount from 3% to 7% (v/v). The squalene oil can be present in an amount from 3% to 7% (v/v). The nonionic surfactant can be present in an amount from 1% to 2.5% (v/v).

In some embodiments, the DL-α-tocopherol can be present in an amount from 0.50% to 4.0% (w/v). The squalene oil can be present in an amount from 0.50% to 4.0% (w/v). The nonionic surfactant can be present in an amount from 0.20% to 2.0% (w/v).

In some embodiments, the immune cells can be human donor peripheral blood immune cells.

In some embodiments, the immune cells can be live T cells.

In some embodiments, the live T cells can be CD4⁺ T cells.

In some embodiments, the live T cells can be CD8⁺ T cells.

In some embodiments, disclosed is a reagent or reagent kit for activating and expanding immune cells in vitro comprising an aqueous solution comprising DL-α-tocopherol, squalene oil, and a nonionic surfactant, and an anti-CD3 and anti-CD28 T-cell activation reagent. The anti-CD3 and anti-CD28 T-cell activation reagent can comprise a plurality of self-assembling protein nanoparticles decorated with anti-CD3 antibodies and anti-CD28 antibodies. At least one of the self-assembling protein nanoparticles can comprise a protein cage polypeptide assembled into a three-dimensional macromolecular structure and can serve as a scaffold for the anti-CD3 antibodies and the anti-CD28 antibodies.

In some embodiments, the three-dimensional macromolecular structure can be a tetrahedral pyramid.

In some embodiments, the DL-α-tocopherol can be present in an amount from 3% to 7% (v/v). The squalene oil can be present in an amount from 3% to 7% (v/v). The nonionic surfactant can be present in an amount from 1% to 2.5% (v/v).

In some embodiments, the DL-α-tocopherol can be present in an amount from 0.50% to 4.0% (w/v). The squalene oil can be present in an amount from 0.50% to 4.0% (w/v). The nonionic surfactant can be present in an amount from 0.20% to 2.0% (w/v).

In some embodiments, the nonionic surfactant can be polysorbate 80.

In some embodiments, the immune cells can be human donor peripheral blood immune cells.

In some embodiments, the immune cells can be live T cells.

In some embodiments, the live T cells can be CD4⁺ T cells.

In some embodiments, the live T cells can be CD8⁺ T cells.

### BRIEF DESCRIPTION OF THE DRAWINGS

FIGS. 1A and 1B are stacked bar graphs illustrating the population of live T cells (CD4⁺ T cells and CD8⁺ T cells) determined using flow cytometry on days 7 and 10, respectively. Quillaja saponins were added to the population of live T cells at the concentrations shown. A STEM-T activated population of live T cells without Quillaja saponins served as the control.

FIGS. 2A and 2B are stacked bar graphs illustrating the population of live T cells (CD4⁺ T cells and CD8⁺ T cells) determined using flow cytometry on days 7 and 10, respectively. QS-21 was added to the population of live T cells at the concentrations shown. A STEM-T activated population of live T cells without QS-21 served as the control.

FIGS. 3A and 3B are stacked bar graphs illustrating the percentage of CD4⁺ T cells relative to CD8⁺ T cells as determined using flow cytometry on days 7 and 10, respectively. Quillaja saponins were added to the population of live T cells at the concentrations shown. A STEM-T activated population of live T cells without Quillaja saponins served as the control.

FIGS. 4A and 4B are stacked bar graphs illustrating the percentage of CD4⁺ T cells relative to CD8⁺ T cells as determined using flow cytometry on days 7 and 10, respectively. QS-21 was added to the population of live T cells at the concentrations shown. A STEM-T activated population of live T cells without QS-21 served as the control.

FIGS. 5A and 5B are stacked bar graphs illustrating the population of live T cells (CD4⁺ T cells and CD8⁺ T cells) determined using flow cytometry on days 7 and 10, respectively. Squalene-based emulsions were added to the population of live T cells at the amounts shown. A STEM-T activated population of live T cells without squalene-based emulsions served as the control.

FIGS. 6A and 6B are stacked bar graphs illustrating the percentage of CD4⁺ T cells relative to CD8⁺ T cells as determined using flow cytometry on days 7 and 10, respectively. Squalene-based emulsions were added to the population of live T cells at the amounts shown. A STEM-T activated population of live T cells without squalene-based emulsions served as the control.

FIGS. 7A and 7B are stacked bar graphs illustrating the population of live T cells (CD4⁺ T cells and CD8⁺ T cells) determined using flow cytometry on days 7 and 10, respectively. QS-21 was added to culture media comprising the population of live T cells at the concentrations shown. A STEM-T activated population of live T cells without QS-21 served as the control.

### DETAILED DESCRIPTION

Disclosed herein are reagents, reagent kits, and culture media for activating and expanding immune cells in vitro. Also disclosed are methods for activating and expanding immune cells in vitro using such reagents, reagent kits, and culture media. The reagents, reagent kits, culture media, and methods disclosed herein can be used as part of a protocol or procedure for manufacturing cell-based immunotherapies.

In some embodiments, a reagent or additive for activating and expanding immune cells in vitro can comprise a Quillaja saponin (e.g., Quil A or QS-21). The Quillaja saponin can be derived from the inner bark or cortex of the Quillaja saponaria Molina tree. For example, the Quillaja saponin can be a water-extractable fraction of saponins from the Quillaja saponaria Molina tree.

In some embodiments, the Quillaja saponin can be dissolved in water. For example, the Quillaja saponin can be initially in a lyophilized form before being dissolved in water.

In some embodiments, the Quillaja saponin can be a purified Quillaja saponin such as QS-21. The QS-21 can initially be in a lyophilized form and then dissolved in phosphate buffered saline (PBS).

QS-21 can have a molecular formula of C₉₂H₁₄₈O₄₆. The Quillaja saponin can have a molecular weight of about 1990.1 g/mol.

For example, QS-21 can have the IUPAC name: (2S,3S,4S,5R,6R)-6-[[(3S,4S,4aR,6aR,6bS,8R,8aR,12aS,14aR,14bR)-8a-[(2S,3R,4S,5R,6R)-3-[(2S,3R,4S,5R,6S)-5-[(2S,3R,4S,5R)-4-[(2S,3R,4R)-3,4-dihydroxy-4-(hydroxymethyl)oxolan-2-yl]oxy-3,5-dihydroxyoxan-2-yl]oxy-3,4-dihydroxy-6-methyloxan-2-yl]oxy-5-[(3S,5S,6S)-5-[(3S,5S,6S)-5-[(2R,3R,4R,5S)-3,4-dihydroxy-5-(hydroxymethyl)oxolan-2-yl]oxy-3-hydroxy-6-methyloctanoyl]oxy-3-hydroxy-6-methyloctanoyl]oxy-4-hydroxy-6-methyloxan-2-yl]oxycarbonyl-4-formyl-8-hydroxy-4,6a,6b,11,11,14b-hexamethyl-1,2,3,4a,5,6,7,8,9,10,12,12a,14,14a-tetradecahydropicen-3-yl]oxy]-3-hydroxy-5-[(2S,3R,4S,5R,6R)-3,4,5-trihydroxy-6-(hydroxymethyl) oxan-2-yl]oxy-4-[(2S,3R,4S,5R)-3,4,5-trihydroxyoxan-2-yl]oxyoxane-2-carboxylic acid.

In other embodiments, the Quillaja saponin can be Quil A.

The immune cells activated and expanded can be human donor peripheral blood immune cells. In some embodiments, the immune cells can be live T cells.

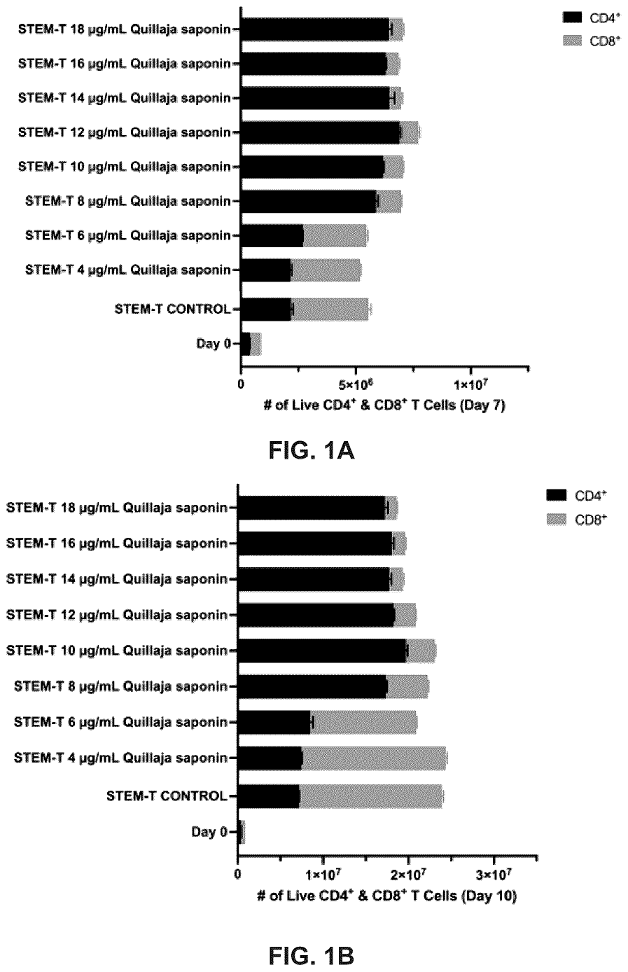

As will be discussed in more detail in the following sections, Applicant discovered that adding reagents or additives comprising Quillaja saponin to immune cells (e.g., T cells) can significantly increase the population of CD4⁺ T cells and change the composition of T cells such that the percentage of CD4⁺ T cells is greatly increased (see FIGS. 1A-1B, 2A-2B, 3A-3B, 4A-4B, and 7A-7B).

Also disclosed is a reagent or reagent kit for activating and expanding immune cells in vitro. The reagent or reagent kit can comprise a solution comprising a Quillaja saponin or a purified Quillaja saponin (e.g., QS-21) and an anti-CD3 and anti-CD28 T-cell activation reagent.

In certain embodiments, the anti-CD3 and anti-CD28 T-cell activation reagent can comprise a plurality of self-assembling protein nanoparticles decorated with anti-CD3 antibodies and anti-CD28 antibodies. The self-assembling protein nanoparticles can comprise protein cage polypeptides assembled into three-dimensional macromolecular structures.

In some embodiments, the three-dimensional macromolecular structure of the protein cage polypeptide can be a tetrahedral pyramid. The protein cage polypeptides can also self-assemble into compact asymmetrical multimeric structures or cage-cage multimers (including dimers).

The three-dimensional macromolecular structures (e.g., protein cage polypeptide formed into tetrahedral pyramids) can serve as scaffolds for the anti-CD3 antibodies and the anti-CD28 antibodies.

The protein cage polypeptide can be any of the protein cage polypeptides or scaffolding proteins discussed in U.S. Patent Publication No. 2022/0196655, the content of which is incorporated herein by reference in its entirety.

In some embodiments, the protein cage polypeptide can be comprised of a polypeptide comprising an amino acid sequence with at least about 70%, 75%, 80%, 85%, 90%, 95%, 99%, or 100% amino acid identity to the amino acid sequence set forth in any one of SEQ ID NOS: 1-3 (see Table 1).

TABLE 1
Sequences of protein cage polypeptides designed and
experimentally tested to-date

| SEQ ID | | |
|---|---|---|
| NO: | NAME | SEQUENCE |
| 1 | Protein cage | MPFITVGQENSTSIDLYYEDHGTGTPVVLIHGFPLSGHSW |
| | polypeptide | RQSAALLDAGYRVITYDRRGFGQSSQPTTGYDYDTFAADE |
| | 1 | LNTVLETLDLQDAVLVGFSMGTGEVARYVSSYGTARIAAV |
| | | AFLASLEPFLLKTDDNPDGAAPQEFFDGIVAAVKADRYAF |
| | | YTGFFNDFYNLDENLGTRISEEAVRNSWNTAASGGFFAAA |
| | | AAPTTWYTDFRADIPRIDVPALILHGTGDRTLPIENTARV |
| | | FHKALPSAEYVEVEGAPHGLLWTHAEEVNTALLAFLAKAQ |
| | | EAQKQKLLTEVETYVLSIIPSGPLKAEIAQRLEDVFAGRW |
| | | GSGADCAWHLGELVWCTAGSGWEDLEVLMEWLKTRPILSP |
| | | LTKGILGFVFTLTVPSERGLQRRRFVQNALNGNGDPNNMD |
| | | KAVKLYRKLKREITFHGAKEISLSYSAGALASCMGLIYNR |
| | | MGAVTTEVAFGLVCATCEQIADSQHRSHRQLEHHHHHH |
| 2 | Protein cage | MPFITVGQENSTSIDLYYEDHGTGTPVVLIHGFPLSGHSW |
| | polypeptide | ERQSAALLDAGYRVITYDRRGFGQSSQPTTGYDYDTFAAD |
| | 2 | LNTVLETLDLQDAVLVGFSMGTGEVARYVSSYGTARIAAV |
| | | AFLASLEPFLLKTDDNPDGAAPQEFFDGIVAAVKADRYAF |
| | | YTGFFNDFYNLDENLGTRISEEAVRNSWNTAASGGFFAAA |
| | | AAPTTWYTDFRADIPRIDVPALILHGTGDRTLPIENTARV |
| | | FHKALPSAEYVEVEGAPHGLLWTHAEEVNTALLAFLAKAQ |
| | | EAQKQKLLTEVETYVLSIIPSGPLKAEIAQRLEDVFAGGG |
| | | RWGADCAWHLGELVWCTAGWEGGDLEVLMEWLKTRPILSP |
| | | LTKGILGFVFTLTVPSERGLQRRRFVQNALNGNGDPNNMD |
| | | KAVKLYRKLKREITFHGAKEISLSYSAGALASCMGLIYNR |
| | | MGAVTTEVAFGLVCATCEQIADSQHRSHRQLEHHHHHH |
| 3 | Protein cage | MPFITVGQENSTSIDLYYEDHGTGTPVVLIHGFPLSGHSW |
| | polypeptide | ERQSAALLDAGYRVITYDRRGFGQSSQPTTGYDYDTFAAD |
| | 3 | LNTVLETLDLQDAVLVGFSMGTGEVARYVSSYGTARIAAV |
| | | AFLASLEPFLLKTDDNPDGAAPQEFFDGIVAAVKADRYAF |
| | | YTGFFNDFYNLDENLGTRISEEAVRNSWNTAASGGFFAAA |
| | | AAPTTWYTDFRADIPRIDVPALILHGTGDRTLPIENTARV |
| | | FHKALPSAEYVEVEGAPHGLLWTHAEEVNTALLAFLAKAQ |
| | | EAQKQKLLTEVETYVLSIIPSGPLKAEIAQRLEDVFAGGA |
| | | DCAWHLGELVWCTAGDLEVLMEWLKTRPILSPLTKGILGF |
| | | VFTLTVPSERGLQRRRFVQNALNGNGDPNNMDKAVKLYRK |
| | | LKREITFHGAKEISLSYSAGALASCMGLIYNRMGAVTTEV |
| | | AFGLVCATCEQIADSQHRSHRQLEHHHHHH |

The protein cage polypeptide can comprise a polypeptide of about 400 to about 700 amino acid residues in length. In some embodiments, the protein cage polypeptide can comprise a polypeptide of about 450 amino acid residues to about 650 amino acid residues in length.

Also disclosed is a culture medium for activating and expanding immune cells in vitro, comprising a Quillaja saponin such as Quil A or QS-21.

In some embodiments, the culture medium can be a T-cell expansion medium. For example, the culture medium can comprise, in addition to the Quillaja saponin (e.g., the Quil A or the QS-21), sodium bicarbonate (NaHCO₃) as a buffer, a visual pH indicator, one or more proteins, supplemental amino acids, carbohydrates, lipids, inorganic salts, enzyme cofactors, vitamins, and (optionally) a zwitterion that can act as an additional buffer.

In certain embodiments, the visual pH indicator can be Phenol Red. The one or more proteins can comprise at least one of albumin, transferrin, fibronectin, and insulin. The supplemental amino acids (e.g., glutamine) can replace those depleted during the cells' growth phase. The carbohydrates can comprise glucose, galactose, maltose, and/or fructose. The lipids or fatty acids can be used by the cells for cell membrane synthesis and activate important signaling pathways. The inorganic salts in the medium (e.g., sodium, potassium, and calcium) can help maintain osmotic balance and regulate membrane potential. The enzyme cofactors can comprise at least one of zinc, copper, selenium, and tricarboxylic acid. The vitamins can comprise riboflavin, thiamine, and/or biotin.

In some embodiments, the culture medium can be a serum-free culture medium. For example, the serum-free culture medium does not contain serum or plasma (although it may contain components derived from serum or plasma such as bovine serum albumin).

In other embodiments, the culture medium can comprise serum or plasma such as fetal bovine serum (FBS).

In some embodiments, the Quillaja saponin such as the Quil A or the QS-21 can be pre-mixed into the culture medium.

In some embodiments, a method of activating and expanding immune cells in vitro can comprise adding the Quillaja saponin (e.g., the Quil A or the QS-21) to a population of immune cells.

The method can further comprise dissolving the Quillaja saponin in water prior to adding the Quillaja saponin to the population of immune cells. The Quillaja saponin can initially be in a lyophilized form.

The method can also comprise dissolving the QS-21 in phosphate buffered saline (PBS) prior to adding the QS-21 to the population of immune cells. The QS-21 can initially be in a lyophilized form.

In some embodiments, the immune cells can be human donor peripheral blood immune cells. For example, the immune cells can be live T cells. As a more specific example, the live T cells activated and expanded can comprise CD4⁺ T cells.

The method can further comprise activating the population of immune cells with an anti-CD3 and anti-CD28 T-cell activation reagent prior to adding the Quillaja saponin (e.g., the Quil A or the QS-21). The T cells can start off as purified CD3⁺ T cells that are activated artificially outside of the body using the anti-CD3 and anti-CD28 T-cell activation reagent.

The anti-CD3 and anti-CD28 T-cell activation reagent can comprise a plurality of self-assembling protein nanoparticles decorated with anti-CD3 antibodies and anti-CD28 antibodies.

In some embodiments, the anti-CD3 antibodies can be the “OKT3” clone with multiple host isotypes, such as human, mouse, rabbit, etc. For example, any of the following anti-CD3 antibodies can be used: (i) anti-CD3 monoclonal antibodies (OKT3) distributed by Takara Bio, (ii) GMP monoclonal anti-human CD3 antibodies (OKT3) distributed by ACROBiosystems, (iii) MACS® GMP CD3 pure antibodies distributed by Miltenyi Biotec, or (iv) GMP Ultra-LEAF™ purified anti-human CD3 SF antibodies distributed by BioLegend.

In some embodiments, the anti-CD28 antibodies can be agonist clones with multiple host isotypes, such as human, mouse, rabbit, etc. For example, any of the following anti-CD28 antibodies can be used: (i) anti-CD28 [YTH 913.12] antibodies distributed by Absolute Antibody, (ii) CD28 antibodies, anti-human, clone 15E8 distributed by Miltenyi Biotec, (iii) Ultra-LEAF™ purified anti-human CD28 antibodies, clone cd28.2, distributed by BioLegend, or (iv) BD™ purified mouse anti-human CD28 antibodies, clone L293, distributed by BD Biosciences.

In some embodiments, a reagent or additive for activating and expanding immune cells in vitro can comprise a squalene-based emulsion. The squalene-based emulsion can comprise DL-α-tocopherol; squalene oil; and a nonionic surfactant.

The DL-α-tocopherol, the squalene oil, and the nonionic surfactant can be combined in an aqueous solution to form the squalene-based emulsion.

In some embodiments, the DL-α-tocopherol is present in an amount from 3% to 7% (v/v), the squalene oil is present in an amount from 3% to 7% (v/v), and the nonionic surfactant is present in an amount from 1% to 2.5% (v/v).

In other embodiments, the DL-α-tocopherol is present in an amount from 0.50% to 4.0% (w/v), the squalene oil is present in an amount from 0.50% to 4.0% (w/v), and the nonionic surfactant is present in an amount from 0.20% to 2.0% (w/v).

Below are three example formulations for the squalene-based emulsion:

Composition 1:

| Component: | Amount: |
|---|---|
| DL-α-tocopherol | 5.0% (v/v) |
| squalene oil | 5.0% (v/v) |
| nonionic surfactant | 1.8% (v/v) |
| (e.g., Polysorbate 80) | |

Composition 2:

| Component: | Amount: |
|---|---|
| DL-α-tocopherol | 2.4% (w/v) |
| squalene oil | 2.2% (w/v) |
| nonionic surfactant | 0.97% (w/v) |
| (e.g., Polysorbate 80) | |

Composition 3:

| Component: | Amount: |
|---|---|
| DL-α-tocopherol | 1.2% (w/v) |
| squalene oil | 1.1% (w/v) |
| nonionic surfactant | 0.49% (w/v) |
| (e.g., Polysorbate 80) | |

In some embodiments, the nonionic surfactant is Polysorbate 80.

The immune cells activated and expanded can be human donor peripheral blood immune cells. In some embodiments, the immune cells can be live T cells.

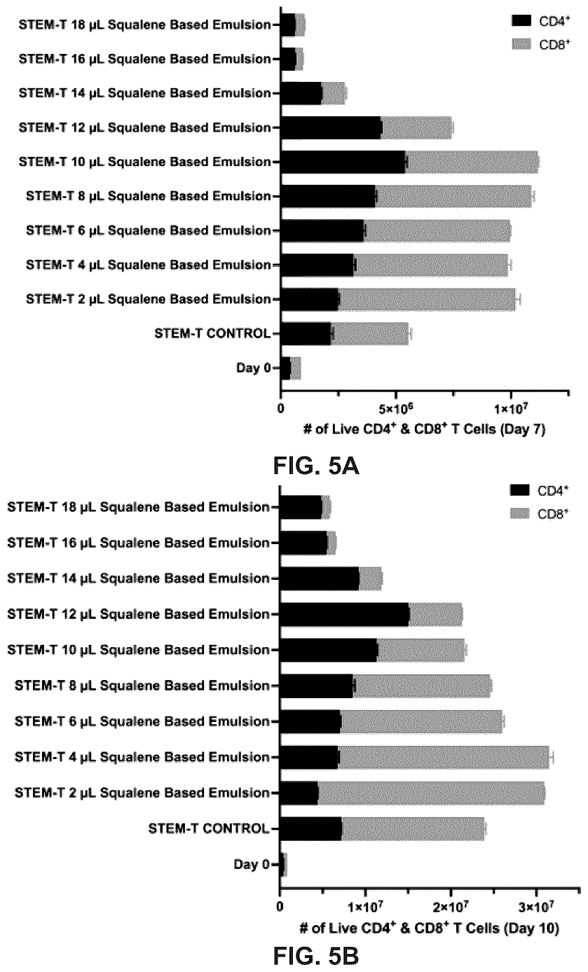

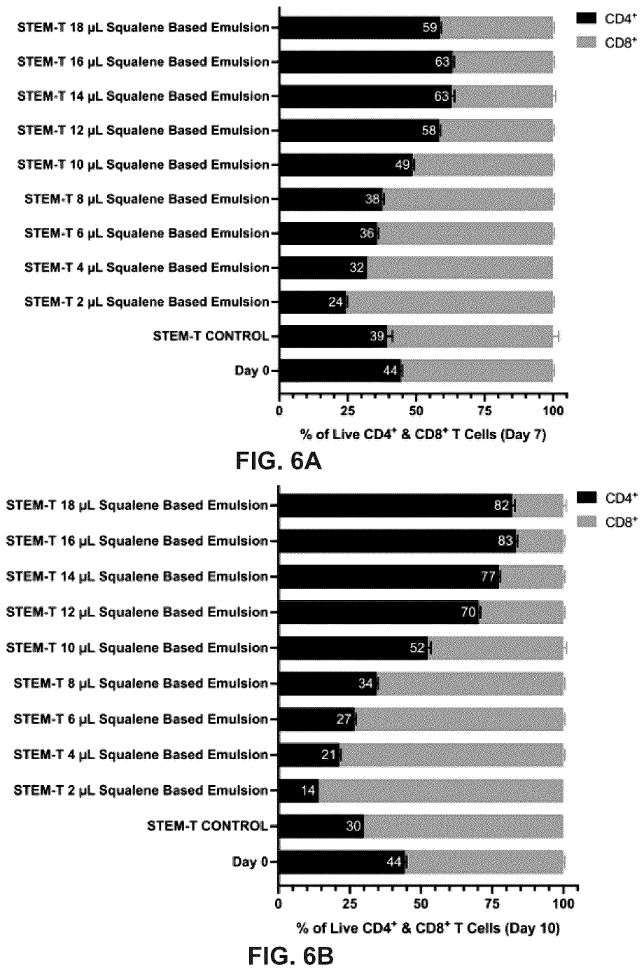

As will be discussed in more detail in the following sections, Applicant discovered that adding reagents or additives comprising the squalene-based emulsion to immune cells (e.g., T cells) can significantly increase the population of CD4⁺ T cells and CD8⁺ T cells in a dose dependent manner. Moreover, the reagent or additive comprising the squalene-based emulsion (e.g., any of Compositions 1-3) can significantly change the composition of T cells such that the percentage of CD4⁺ T cells and/or the percentage of CD8⁺ T cells are greatly increased (see FIGS. 5A-5B and 6A-6B).

Also disclosed is a reagent or reagent kit for activating and expanding immune cells in vitro. The reagent can comprise the squalene-based emulsion (e.g., any of Compositions 1-3) and the anti-CD3 and anti-CD28 T-cell activation reagent.

As previously discussed, the anti-CD3 and anti-CD28 T-cell activation reagent can comprise a plurality of self-assembling protein nanoparticles decorated with anti-CD3 antibodies and anti-CD28 antibodies. The self-assembling protein nanoparticles can comprise protein cage polypeptides assembled into three-dimensional macromolecular structures (e.g., tetrahedral pyramids).

In some embodiments, the anti-CD3 antibodies can be the “OKT3” clone with multiple host isotypes, such as human, mouse, rabbit, etc. For example, any of the following anti-CD3 antibodies can be used: (i) anti-CD3 monoclonal antibodies (OKT3) distributed by Takara Bio, (ii) GMP monoclonal anti-human CD3 antibodies (OKT3) distributed by ACROBiosystems, (iii) MACS® GMP CD3 pure antibodies distributed by Miltenyi Biotec, or (iv) GMP Ultra-LEAF™ purified anti-human CD3 SF antibodies distributed by BioLegend.

In some embodiments, the anti-CD28 antibodies can be agonist clones with multiple host isotypes, such as human, mouse, rabbit, etc. For example, any of the following anti-CD28 antibodies can be used: (i) anti-CD28 [YTH 913.12] antibodies distributed by Absolute Antibody, (ii) CD28 antibodies, anti-human, clone 15E8 distributed by Miltenyi Biotec, (iii) Ultra-LEAF™ purified anti-human CD28 antibodies, clone cd28.2, distributed by BioLegend, or (iv) BD™ purified mouse anti-human CD28 antibodies, clone L293, distributed by BD Biosciences.

Also disclosed is a culture medium for activating and expanding immune cells in vitro, comprising the squalene-based emulsion.

In some embodiments, the culture medium can be a T-cell expansion medium. For example, the culture medium can comprise, in addition to the squalene-based emulsion, sodium bicarbonate (NaHCO₃) as a buffer, a visual pH indicator, one or more proteins, supplemental amino acids, carbohydrates, lipids, inorganic salts, enzyme cofactors, vitamins, and (optionally) a zwitterion that can act as an additional buffer.

In certain embodiments, the visual pH indicator can be Phenol Red. The one or more proteins can comprise at least one of albumin, transferrin, fibronectin, and insulin. The supplemental amino acids (e.g., glutamine) can replace those depleted during the cells' growth phase. The carbohydrates can comprise glucose, galactose, maltose, and/or fructose. The lipids or fatty acids can be used by the cells for cell membrane synthesis and activate important signaling pathways. The inorganic salts in the medium (e.g., sodium, potassium, and calcium) can help maintain osmotic balance and regulate membrane potential. The enzyme cofactors can comprise at least one of zinc, copper, selenium, and tricarboxylic acid. The vitamins can comprise riboflavin, thiamine, and/or biotin.

In some embodiments, the culture medium can be a serum-free culture medium. For example, the serum-free culture medium does not contain serum or plasma (although it may contain components derived from serum or plasma such as bovine serum albumin).

In other embodiments, the culture medium can comprise serum or plasma such as fetal bovine serum (FBS).

In some embodiments, the squalene-based emulsion can be pre-mixed into the culture medium.

In some embodiments, a method of activating and expanding immune cells in vitro can comprise adding an aqueous solution comprising DL-α-tocopherol, squalene oil, and a nonionic surfactant (e.g., any of Compositions 1-3) to a population of immune cells.

In some embodiments, the immune cells can be human donor peripheral blood immune cells. For example, the immune cells can be live T cells. As a more specific example, the live T cells activated and expanded can comprise CD4⁺ T cells and CD8⁺ T cells.

The method can further comprise comprising activating the population of immune cells with the anti-CD3 and anti-CD28 T-cell activation reagent prior to adding the aqueous solution comprising the DL-α-tocopherol, squalene oil, and nonionic surfactant (the squalene-based emulsion). The T cells can start off as purified CD3⁺ T cells that are activated artificially outside of the body using the anti-CD3 and anti-CD28 T-cell activation reagent.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1: Quillaja Saponins Enhance T-Cell Activation and Expansion In Vitro

One unexpected result stemming from the experiments disclosed herein is that Quillaja saponins can increase T-cell activation and expansion in vitro when compared to T-cell activation with a T-cell activation reagent alone.

Human peripheral blood CD3⁺ T cells were thawed and media exchanged in serum free T-cell expansion medium on day 0. T cells were seeded at a concentration of 1×10⁶ cells/mL then placed in a 37° C. and 5% CO₂ incubator for approximately 4 hours. The T cells were then activated with an anti-CD3 and anti-CD28 T-cell activation reagent. The T-cell activation reagent is referred to herein and shown in the figures as STEM-T.

The STEM-T activation reagent comprises a plurality of self-assembling protein nanoparticles decorated with anti-CD3 antibodies and anti-CD28 antibodies. The self-assembling protein nanoparticles comprise protein cage polypeptides assembled into three-macromolecular dimensional structures. Each of the three-dimensional macromolecular structures can serve as a scaffold for the anti-CD3 antibodies and the anti-CD28 antibodies.

In some embodiments, the three-dimensional macromolecular structure formed by the protein cage polypeptide can be a tetrahedral pyramid. The protein cage polypeptides can also self-assemble into compact asymmetrical multimeric structures or cage-cage multimers (including dimers).

Different concentrations of a Quillaja saponin and a purified Quillaja saponin, QS-21, were added to the population of live T cells after activating the live T cells with STEM-T. A STEM-T activated population of live T cells without saponins served as the control. Cytokines (e.g., IL-7 and IL-15) were then added to the medium and then returned to the 37° C. and 5% CO₂ incubator. For example, IL-7 and IL-15 were added to a final concentration of 10 ng/ml. The media was exchanged every ˜3 days with fresh media supplemented with cytokines.

FIGS. 1A and 1B are stacked bar graphs illustrating the population of live T cells (CD4⁺ T cells and CD8⁺ T cells) determined using flow cytometry on days 7 and 10, respectively. Quillaja saponins were added to the population of live T cells shown in FIGS. 1A and 1B at the following concentrations after activating the live T cells with STEM-T: 4 μg/mL, 6 g/mL, 8 μg/mL, 10 μg/mL, 12 μg/mL, 14 μg/mL, 16 μg/mL, and 18 μg/mL (where the concentrations refer to μg of Quillaja saponins per mL of culture medium). The Quillaja saponin used was Quil A. Quil A saponins are commercially available under the brand name Quil-AR distributed by InvivoGen.

It is important to note that “Quil A” when used in this application refers to a mixture of triterpenoid saponins isolated from an extract of the Quillaja saponaria bark. The mixture of triterpenoid saponins in Quil A contains at least the fractions QS-17, QS-18, and QS-21. The mixture name “Quil A” has been used in the field since at least the 1970s. Sec Dalsgaard, K., Merethe H. Jensen, and K. J. Sørensen. Saponin adjuvants. IV. Evaluation of the adjuvant Quil A in the vaccination of cattle against foot-and-mouth disease. Acta veterinaria scandinavica 18.3 (1977): 349-360, the content of which is incorporated herein by reference in its entirety. The mixture name “Quil A” predates the registered trademark Quil-AR by several decades (the U.S. trademark for Quil-AR registered on Jun. 2, 2015). Any references to Quil A in this application (and the claims of this application) refers to the mixture of triterpenoid saponins isolated from an extract of the Quillaja saponaria bark. This mixture of triterpenoid saponins is also commonly referred to as Quil A in the scientific literature.

For example, the Quil A saponin initially started off as a lyophilized powder. The saponin powder was dissolved in deionized water to a working stock concentration of 10 mg/mL. The working stock concentration was then diluted to the aforementioned concentrations by being added into the culture medium (e.g., T-cell expansion medium).

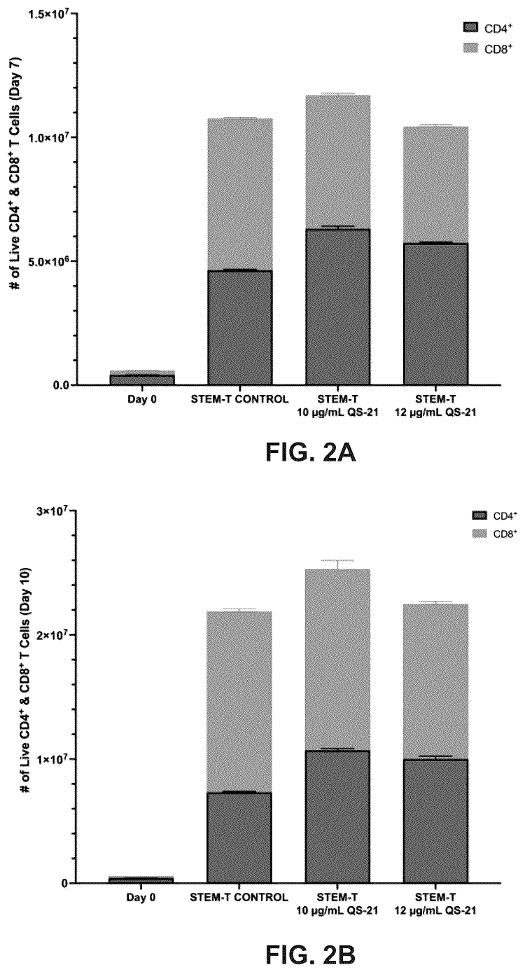

FIGS. 2A and 2B are stacked bar graphs illustrating the population of live T cells (CD4⁺ T cells and CD8⁺ T cells) determined using flow cytometry on days 7 and 10, respectively. QS-21 was added to the population of live T cells shown in FIGS. 2A and 2B at concentrations of 10 μg/mL and 12 μg/mL (where the concentrations refer to μg of QS-21 per mL of culture medium) after activating the live T cells with STEM-T. The QS-21 used was distributed by MedChemExpress.

For example, the QS-21 initially started off as a lyophilized film. The lyophilized film was dissolved in phosphate buffered saline (PBS) to a working stock concentration of 5 mg/mL. The working stock concentration was then diluted to the aforementioned concentrations by being added into the culture medium (e.g., T-cell expansion medium).

As can be seen from FIGS. 1A-1B and 2A-2B, the total number of live T cells (CD4⁺ T cells and CD8⁺ T cells combined) increased when compared to the STEM-T control in response to the addition of 10 μg/mL of QS-21 (as measured on days 7 and 10) and the addition of at least 8 μg/mL of the Quillaja saponin (as measured on day 7).

Moreover, another unexpected result stemming from the experiments disclosed herein is that the number of live CD4⁺ T cells increased significantly when compared to the STEM-T control in response to the addition of at least 8 μg/mL of Quillaja saponin and the addition of 10 μg/mL of QS-21 (as measured on days 7 and 10).

Furthermore, yet another unexpected result stemming from the experiments disclosed herein is that Quillaja saponins, including purified Quillaja saponins (e.g., QS-21), at concentrations between 10 μg/mL and 12 μg/mL of media enhanced T-cell expansion compared to activated controls.

For example, QS-21 can be purified using reverse-phase chromatography (RP-HPLC). QS denotes Quillaja saponaria and the number 21 is the identity of the RP-HPLC peak [7]. Methods for purifying QS-21 from the inner bark or cortex of the Quillaja saponaria Molina tree using silica and RP-HPLC are known in the art [8].

### Example 2: Quillaja Saponins Change the Composition of T Cells Expanded In Vitro

One unexpected result stemming from the experiments disclosed herein is that Quillaja saponins can change the composition of T cells such that the percentage of CD4⁺ T cells is greatly increased.

Flow cytometry was also used to determine the percentage of CD4⁺ T cells relative to CD8⁺ T cells for the T cells expanded using the method discussed in Example 1.

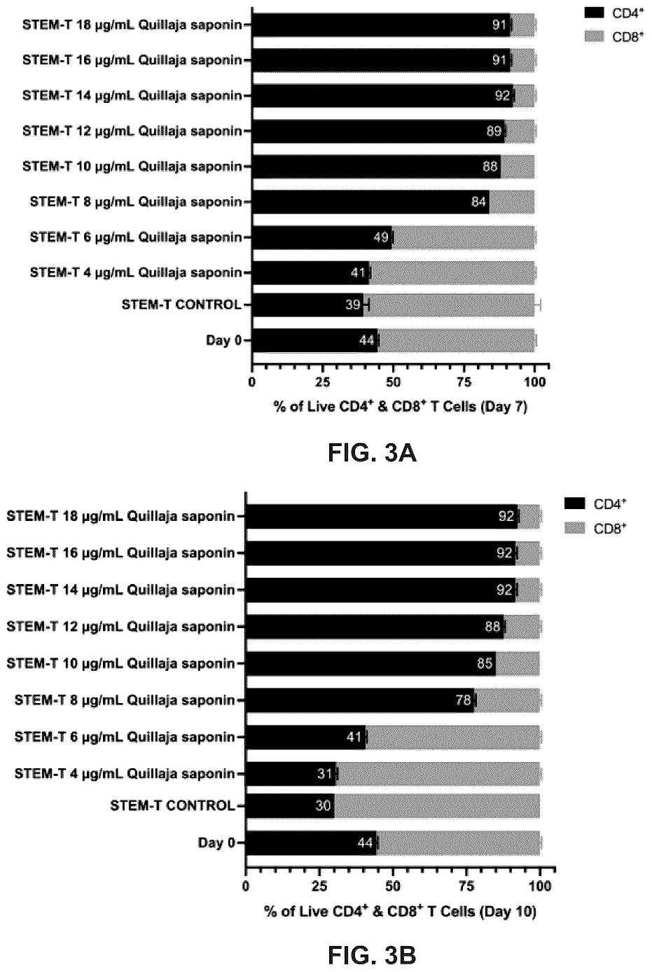

FIGS. 3A and 3B are stacked bar graphs illustrating the percentage of CD4⁺ T cells relative to CD8⁺ T cells as determined using flow cytometry on days 7 and 10, respectively. Quillaja saponins were added to the population of live T cells shown in FIGS. 3A and 3B at the following concentrations after activating the live T cells with STEM-T: 4 μg/mL, 6 μg/mL, 8 μg/mL, 10 μg/mL, 12 μg/mL, 14 μg/mL, 16 μg/mL, and 18 μg/mL. The Quillaja saponin used was Quil A. Quil A saponins are commercially available under the brand name Quil-AR distributed by InvivoGen.

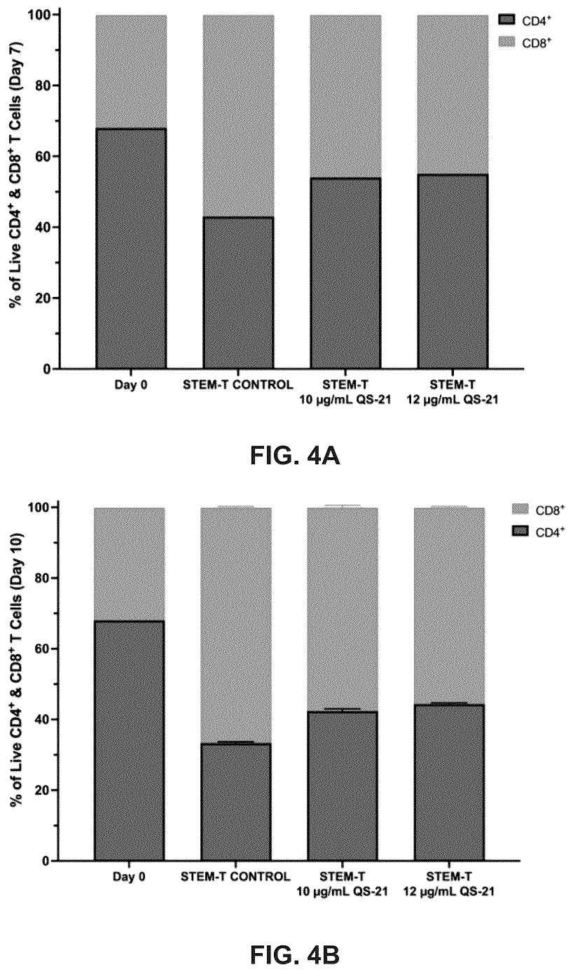

FIGS. 4A and 4B are stacked bar graphs illustrating the percentage of CD4⁺ T cells relative to CD8⁺ T cells as determined using flow cytometry on days 7 and 10, respectively. QS-21 was added to the population of live T cells shown in FIGS. 4A and 4B at concentrations of 10 μg/mL and 12 μg/mL after activating the live T cells with STEM-T. The QS-21 used was distributed by MedChemExpress.

In studies, QS-21 has been shown to induce a combination of a Th1 (cell-mediated immunity including phagocyte-dependent response) and Th2 (antibody production and eosinophil activation) immune response [9].

As can be seen from FIGS. 3A-3B, the percentage of CD4⁺ T cells relative to CD8⁺ T cells increased significantly when compared to the STEM-T control in response to the addition of at least 8 μg/mL of Quillaja saponin (as measured on days 7 and 10).

Moreover, as can be seen from FIGS. 4A-4B, the percentage of CD4⁺ T cells relative to CD8⁺ T cells increased when compared to the STEM-T control in response to the addition of at least 10 μg/mL of QS-21.

### Example 3: Squalene-Based Emulsions Enhance T-Cell Activation and Expansion In Vitro

One unexpected result stemming from the experiments disclosed herein is that squalene-based emulsions can increase T-cell activation and expansion in vitro when compared to T-cell activation with a T-cell activation reagent alone.

Human peripheral blood CD3⁺ T cells were thawed and media exchanged in serum free T-cell expansion medium on day 0. T cells were seeded at a concentration of 1×10⁶ cells/mL then placed in a 37° C. and 5% CO₂ incubator for approximately 4 hours. The T cells were then activated with an anti-CD3 and anti-CD28 T-cell activation reagent. The T-cell activation reagent is referred to herein and shown in the figures as STEM-T.

Different concentrations of a squalene-based emulsion were added to the population of live T cells after activating the live T cells with STEM-T. A STEM-T activated population of live T cells without the squalene-based emulsion served as the control.

In some embodiments, the anti-CD3 antibodies can be the “OKT3” clone with multiple host isotypes, such as human, mouse, rabbit, etc. For example, any of the following anti-CD3 antibodies can be used: (i) anti-CD3 monoclonal antibodies (OKT3) distributed by Takara Bio, (ii) GMP monoclonal anti-human CD3 antibodies (OKT3) distributed by ACROBiosystems, (iii) MACS® GMP CD3 pure antibodies distributed by Miltenyi Biotec, or (iv) GMP Ultra-LEAF™ purified anti-human CD3 SF antibodies distributed by BioLegend.

In some embodiments, the anti-CD28 antibodies can be agonist clones with multiple host isotypes, such as human, mouse, rabbit, etc. For example, any of the following anti-CD28 antibodies can be used: (i) anti-CD28 [YTH 913.12] antibodies distributed by Absolute Antibody, (ii) CD28 antibodies, anti-human, clone 15E8 distributed by Miltenyi Biotec, (iii) Ultra-LEAF™ purified anti-human CD28 antibodies, clone cd28.2, distributed by BioLegend, or (iv) BD™ purified mouse anti-human CD28 antibodies, clone L293, distributed by BD Biosciences.

Cytokines were added to the medium and then returned to the 37° C. and 5% CO₂ incubator. The media was exchanged every ˜3 days with fresh media supplemented with cytokines.

FIGS. 5A and 5B are stacked bar graphs illustrating the population of live T cells (CD4⁺ T cells and CD8⁺ T cells) determined using flow cytometry on days 7 and 10, respectively. Squalene-based emulsions were added to the population of live T cells at the following volumes after activating the live T cells with STEM-T: 2 μL, 4 μL, 6 μL, 8 μL, 10 μL, 12 μL, 14 μL, 16 μL, and 18 μL. The squalene-based emulsion used was AddaS03™ distributed by InvivoGen.

AddaS03™ is similar to Adjuvant System 03 (AS03), which is an adjuvant containing α-tocopherol and squalene in an oil-in-water emulsion. In clinical trials in humans, AS03 was shown to cause B cell memory, antibody responses, and CD4+ T cell responses, along with cytokine production [10].

As can be seen from FIGS. 5A-5B, the total number of live T cells (CD4⁺ T cells and CD8⁺ T cells combined) increased when compared to the STEM-T control in response to the addition of between 2 μL and 8 μL of the squalene-based emulsion (as measured on both days 7 and 10) and the addition of between 10 μL and 12 μL of the squalene-based emulsion (as measured on day 7).

Moreover, another unexpected result stemming from the experiments disclosed herein is that the number of live CD8⁺ T cells increased significantly when compared to the STEM-T control in response to the addition of between 2 μL and 10 μL of the squalene-based emulsion, as measured on day 7, and the addition of between 2 μL and 4 μL of the squalene-based emulsion, as measured on day 10.

Furthermore, yet another unexpected result stemming from the experiments disclosed herein is that the number of live CD4⁺ T cells increased significantly when compared to the STEM-T control in response to the addition of between 10 μL and 12 μL of the squalene-based emulsion, as measured on days 7 and 10. Moreover, as can be seen in FIGS. 5A and 5B, the number of live CD4⁺ T cells increased when compared to the STEM-T control in response to the addition of between 2 μL and 12 μL of the squalene-based emulsion, as measured on day 7, and the addition of between 8 μL and 14 μL of the squalene-based emulsion, as measured on day 10.

### Example 4: Squalene-Based Emulsions Change the Composition of T Cells Expanded In Vitro

One unexpected result stemming from the experiments disclosed herein is that squalene-based emulsions can change the composition of T cells such that the percentages of CD4⁺ T cells and CD8⁺ T cells can be modulated.

Flow cytometry was also used to determine the percentage of CD4⁺ T cells relative to CD8⁺ T cells for the T cells expanded using the method discussed in Example 3.

FIGS. 6A and 6B are stacked bar graphs illustrating the percentage of CD4⁺ T cells relative to CD8⁺ T cells as determined using flow cytometry on days 7 and 10, respectively.

Squalene-based emulsions were added to the population of live T cells at the following volumes after activating the live T cells with STEM-T: 2 μL, 4 μL, 6 μL, 8 μL, 10 μL, 12 μL, 14 μL, 16 μL, and 18 μL. The squalene-based emulsion used was AddaS03™ distributed by InvivoGen.

As can be seen from FIGS. 6A and 6B, the percentage of CD8⁺ T cells increased when compared to the STEM-T control in response to the addition of between 2 μL and 8 μL of the squalene-based emulsion, as measured on day 7, and the addition of between 2 μL and 6 μL of the squalene-based emulsion, as measured on day 10.

Moreover, as can be seen from FIGS. 6A and 6B, the percentage of CD4⁺ T cells increased when compared to the STEM-T control in response to the addition of between 10 μL and 18 μL of the squalene-based emulsion, as measured on day 7, and the addition of between 8 μL and 18 μL of the squalene-based emulsion, as measured on day 10. The percentage of CD4⁺ T cells increased significantly when compared to the STEM-T control when at least 10 μL of the squalene-based emulsion was added.

### Example 5: QS-21 Changes the Composition of T Cells Expanded In Vitro

Another unexpected result stemming from the experiments disclosed herein is that QS-21, even at small concentrations, can change the composition of T cells such that the percentage of CD4⁺ T cells is greatly increased relative to CD8⁺ T cells.

The T cells were prepared by thawing human peripheral blood CD3⁺ T cells and the cell medium was exchanged with T cell medium. For example, the T cell medium used can comprise salts, sugars, amino acids, vitamins, and/or buffers. As a more specific example, the T cell culture medium can be the CellGenix® GMP T cell culture medium (datasheet available at https://download.dam.sartorius.com/d/asset/295401/CGX-GMP-TCM-Datasheet-en-sartorius.pdf, the content of which is incorporated herein by reference in its entirety). T cells were seeded (e.g., at a concentration of 1×10⁶ cells/mL) then placed in a 37° C. and 5% CO₂ incubator for approximately 4 hours. A reagent was then added to 1 mL of the T cell culture medium comprising the CD4⁺ T cells and the CD8⁺ T cells. The reagent comprised QS-21 in solution and the anti-CD3 and anti-CD28 T-cell activation reagent.

The anti-CD3 and anti-CD28 T-cell activation reagent is also referred to as the STEM-T activation reagent. The STEM-T activation reagent comprises a plurality of self-assembling protein nanoparticles bound to or decorated with anti-CD3 antibodies and anti-CD28 antibodies. Each of the self-assembling protein nanoparticles can comprise polypeptides assembled into a three-dimensional macromolecular structure. For example, each of the three-dimensional macromolecular structures can serve as a scaffold for the anti-CD3 antibodies and the anti-CD28 antibodies. In some embodiments, the three-dimensional macromolecular structure formed by the protein cage polypeptide can be a tetrahedral pyramid. The protein cage polypeptides can also self-assemble into compact asymmetrical multimeric structures or cage-cage multimers (including dimers).

The reagent was added to the T cell culture media in various volumes to cause the final QS-21 concentration to be 1.5 μg/mL, 1.8 μg/mL, 2.1 μg/mL, 2.4 μg/mL, 2.7 μg/mL, 3.0 μg/mL, 3.3 μg/mL, 3.6 μg/mL, and 3.9 μg/mL (where the concentrations refer to μg of QS-21 per mL of culture medium). For example, 5, 6, 7, 8, 9, 10, 11, 12, and 13 μL of the reagent were added to 1 mL of the T cell culture medium comprising the CD4⁺ T cells and the CD8⁺ T cells. The reagent comprised 300 μg/mL of QS-2 and the anti-CD3 and anti-CD28 T-cell activation reagent.

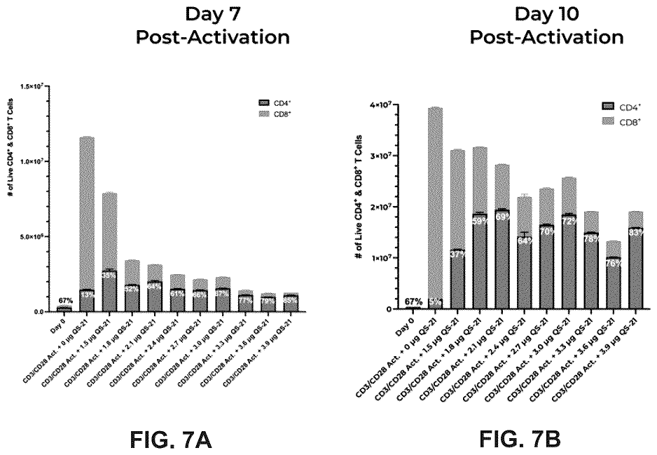

FIGS. 7A and 7B are stacked bar graphs illustrating the percentage of CD4⁺ T cells relative to CD8⁺ T cells as determined using flow cytometry on days 7 and 10, respectively.

As can be seen from FIGS. 7A and 7B, the percentage of CD4⁺ T cells (as indicated by the listed percentages) relative to CD8⁺ T cells increased significantly when compared to the STEM-T control, which is indicated as “CD3/CD28 Act.+0 μg QS-21.”

Another surprising discovery is that the total number of live T cells (CD4⁺ T cells and CD8⁺ T cells) decreased relative to the STEM-T control (especially seven days post-activation). Ten days post-activation, the total number of live T cells increased and the percentage of CD4⁺ T cells relative to CD8⁺ T cells increased significantly when compared to the STEM-T control. This increase in the percentage of CD4⁺ T cells and decrease in the percentage of CD8⁺ T cells can be seen in response to the addition of at least 1.5 μg/mL of QS-21.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit, or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications, and journal articles) are incorporated by reference herein in their entireties except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of” when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially,” “about,” and “approximately” as used herein mean the specified value or the specified value and a reasonable amount of deviation from the specified value (e.g., a deviation of up to +0.1%, +1%, +5%, or +10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

The structures in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

All cited references are hereby incorporated by reference in their entireties.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

### REFERENCES

[1] Centers for Disease Control and Prevent, Adjuvants and Vaccines. Retrieved Jul. 9, 2023 from https://www.cdc.gov/vaccinesafety/concerns/adjuvants.html.
[2] Shi, S. et al. Vaccine adjuvants: Understanding the structure and mechanism of adjuvanticity. Vaccine 37, 3167-3178 (2019).
[3] Hearnden, Claire, and Ed C. Lavelle. Adjuvant strategies for vaccines: the use of adjuvants within the cancer vaccine setting. Cancer Immunotherapy. Academic Press, 333-349 (2013).
[4] Singh, M. & O'Hagan, D. T. Recent advances in veterinary vaccine adjuvants. Int. J. Parasitol. 33, 469-478 (2003).
[5] Wang, P. Natural and Synthetic Saponins as Vaccine Adjuvants. Vaccines (Basel) 9, 222 (2021).
[6] Lacaille-Dubois, Marie-Aleth. Updated insights into the mechanism of action and clinical profile of the immunoadjuvant QS-21: A review. Phytomedicine 60: 152905 (2019).
[7] Zhu, Daming, and Wenbin Tuo. QS-21: a potent vaccine adjuvant. Natural Products Chemistry & Research 3.4 (2016).
[8] Kensil, Charlotte R., et al. Separation and characterization of saponins with adjuvant activity from Quillaja saponaria Molina cortex. Journal of Immunology 146.2: 431-437 (1991).
[9] Romagnani, Sergio. Th1/th2 cells. Inflammatory bowel diseases 5.4: 285-294 (1999).
[10] Morel, Sandra, et al. Adjuvant System AS03 containing α-tocopherol modulates innate immune response and leads to improved adaptive immunity. Vaccine 29.13: 2461-2473 (2011).

We claim:

1. A reagent for activating and expanding CD4⁺ T cells and CD8⁺ T cells in vitro, comprising:
a Quillaja saponin in solution, wherein the Quillaja saponin when added to a culture medium comprising the CD4⁺ T cells and the CD8⁺ T cells is at a concentration between 1.5 μg/mL and 3.9 μg/mL, wherein the μg/mL is μg of the Quillaja saponin per mL of the culture medium, wherein the Quillaja saponin comprises QS-21; and
an activation reagent compromising a plurality of self-assembling protein nanoparticles bound to anti-CD3 antibodies and anti-CD28 antibodies, wherein the plurality of self-assembling protein nanoparticles comprises polypeptides assembled into a three-dimensional macromolecular structure.

2. The reagent of claim 1, wherein the CD4⁺ T cells and the CD8⁺ T cells are derived from CD3⁺ human peripheral blood cells.

3. The reagent of claim 1, wherein the self-assembling nanoparticles comprises protein cage polypeptides assembled into the three-dimensional macromolecular structure, and wherein at least one of the protein cage polypeptides comprises an amino acid sequence with at least about 70% identity to at least one of SEQ ID NO:1, SEQ ID NO:2, and SEQ ID NO:3.

4. A reagent for activating and expanding CD4⁺ T cells and CD8⁺ T cells in vitro, comprising:
a Quillaja saponin in solution, wherein the Quillaja saponin when added to a culture medium comprising the CD4⁺ T cells and the CD8⁺ T cells is at a concentration between 8 μg/mL and 18 μg/mL, wherein the μg/mL is μg of the Quillaja saponin per mL of the culture medium, wherein the Quillaja saponin is Quil A; and
an activation reagent compromising a plurality of self-assembling protein nanoparticles bound to anti-CD3 antibodies and anti-CD28 antibodies, wherein the plurality of self-assembling protein nanoparticles comprises polypeptides assembled into a three-dimensional macromolecular structure.

5. The reagent of claim 4, wherein the CD4⁺ T cells and the CD8⁺ T cells are derived from CD3⁺ human peripheral blood cells.

6. The reagent of claim 4, wherein the self-assembling nanoparticles comprises protein cage polypeptides assembled into the three-dimensional macromolecular structure, and wherein at least one of the protein cage polypeptides comprises an amino acid sequence with at least about 70% identity to at least one of SEQ ID NO:1, SEQ ID NO:2, and SEQ ID NO:3.

7. A method of activating and expanding CD4⁺ T cells and CD8⁺ T cells in vitro, comprising:
adding a reagent to a culture medium comprising CD4⁺ T cells and CD8⁺ T cells, wherein the reagent comprises:
a Quillaja saponin in solution, wherein a concentration of the Quillaja saponin added to the culture medium is between 1.5 μg/mL and 3.9 μg/mL, wherein the μg/mL is μg of the Quillaja saponin per mL of the culture medium, wherein the Quillaja saponin comprises QS-21; and
an activation reagent compromising a plurality of self-assembling protein nanoparticles bound to anti-CD3 antibodies and anti-CD28 antibodies, wherein the plurality of self-assembling protein nanoparticles comprises polypeptides assembled into a three-dimensional macromolecular structure.

8. The method of claim 7, wherein the CD4⁺ T cells and the CD8⁺ T cells are derived from CD3⁺ human peripheral blood cells.

9. The method of claim 7, wherein the self-assembling nanoparticles comprises protein cage polypeptides assembled into the three-dimensional macromolecular structure, and wherein at least one of the protein cage polypeptides comprises an amino acid sequence with at least about 70% identity to at least one of SEQ ID NO:1, SEQ ID NO:2, and SEQ ID NO:3.

10. A method of activating and expanding CD4⁺ T cells and CD8⁺ T cells in vitro, comprising:
adding a reagent to a culture medium comprising CD4⁺ T cells and CD8⁺ T cells, wherein the reagent comprises:
a Quillaja saponin in solution, wherein a concentration of the Quillaja saponin added to the culture medium is between 8 μg/mL and 18 μg/mL, wherein the μg/mL is μg of the Quillaja saponin per mL of the culture medium, wherein the Quillaja saponin comprises QS-21; and
an activation reagent compromising a plurality of self-assembling protein nanoparticles bound to anti-CD3 antibodies and anti-CD28 antibodies, wherein the plurality of self-assembling protein nanoparticles comprises polypeptides assembled into a three-dimensional macromolecular structure.

11. A method of activating and expanding CD4⁺ T cells and CD8⁺ T cells in vitro, comprising:
adding a reagent to a culture medium comprising CD4⁺ T cells and CD8⁺ T cells, wherein the reagent comprises:
a Quillaja saponin in solution, wherein a concentration of the Quillaja saponin added to the culture medium is between 8 μg/mL and 18 μg/mL, wherein the μg/mL is μg of the Quillaja saponin per mL of the culture medium, wherein the Quillaja saponin is Quil A; and
an activation reagent, compromising a plurality of self-assembling protein nanoparticles bound to anti-CD3 antibodies and anti-CD28 antibodies, wherein the plurality of self-assembling protein nanoparticles comprises polypeptides assembled into a three-dimensional macromolecular structure.

12. The method of claim 11, wherein the CD4⁺ T cells and the CD8⁺ T cells are derived from CD3⁺ human peripheral blood cells.

13. The method of claim 11, wherein the self-assembling nanoparticles comprises protein cage polypeptides assembled into the three-dimensional macromolecular structure, and wherein at least one of the protein cage polypeptides comprises an amino acid sequence with at least about 70% identity to at least one of SEQ ID NO:1, SEQ ID NO:2, and SEQ ID NO:3.

14. A reagent for activating and expanding CD4⁺ T cells and CD8⁺ T cells in vitro, comprising:
DL-α-tocopherol;
squalene oil;
a nonionic surfactant; and
an anti-CD3 and anti-CD28 T-cell activation reagent, comprising:
a plurality of self-assembling protein nanoparticles decorated with anti-CD3 antibodies and anti-CD28 antibodies, wherein at least one of the self-assembling protein nanoparticles comprises polypeptides assembled into a three-dimensional macromolecular structure and serving as a scaffold for the anti-CD3 antibodies and the anti-CD28 antibodies.

15. The reagent of claim 14, wherein the three-dimensional macromolecular structure is a tetrahedral pyramid.

16. The reagent of claim 14, wherein the DL-α-tocopherol is present in an amount from 3% to 7% (v/v), wherein the squalene oil is present in an amount from 3% to 7% (v/v), and wherein the nonionic surfactant is present in an amount from 1% to 2.5% (v/v).

17. The reagent of claim 14, wherein the DL-α-tocopherol is present in an amount from 0.50% to 4.0% (w/v), wherein the squalene oil is present in an amount from 0.50% to 4.0% (w/v), and wherein the nonionic surfactant is present in an amount from 0.20% to 2.0% (w/v).

18. The reagent of claim 14, wherein the nonionic surfactant is polysorbate 80.

19. The reagent of claim 14, wherein the CD4⁺ T cells and the CD8⁺ T cells are derived from CD3⁺ human peripheral blood cells.

20. A method of activating and expanding CD4⁺ T cells and CD8⁺ T cells in vitro, comprising:
adding a reagent to a culture medium comprising CD4⁺ T cells and CD8⁺ T cells, wherein the reagent comprises:
an aqueous solution comprising DL-α-tocopherol, squalene oil, and a nonionic surfactant; and
an activation reagent compromising a plurality of self-assembling protein nanoparticles bound to anti-CD3 antibodies and anti-CD28 antibodies, wherein the plurality of self-assembling protein nanoparticles comprises polypeptides assembled into a three-dimensional macromolecular structure.


SEQUENCE LISTING (3 sequences)

SEQ ID NO: 1 - protein, 478 aa, synthetic construct
MPFITVGQENSTSIDLYYEDHGTGTPVVLIHGFPLSGHSWERQSAALLDAGYRVITYDRRGFGQSSQPTTGYDYDTFAADLNTVLETLDLQDAVLVGFSMGTGEVARYVSSYGTARIAAVAFLASLEPFLLKTDDNPDGAAPQEFFDGIVAAVKADRYAFYTGFFNDFYNLDENLGTRISEEAVRNSWNTAASGGFFAAAAAPTTWYTDFRADIPRIDVPALILHGTGDRTLPIENTARVFHKALPSAEYVEVEGAPHGLLWTHAEEVNTALLAFLAKAQEAQKQKLLTEVETYVLSIIPSGPLKAEIAQRLEDVFAGRWGSGADCAWHLGELVWCTAGSGWEDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQLEHHHHHH

SEQ ID NO: 2 - protein, 478 aa, synthetic construct
MPFITVGQENSTSIDLYYEDHGTGTPVVLIHGFPLSGHSWERQSAALLDAGYRVITYDRRGFGQSSQPTTGYDYDTFAADLNTVLETLDLQDAVLVGFSMGTGEVARYVSSYGTARIAAVAFLASLEPFLLKTDDNPDGAAPQEFFDGIVAAVKADRYAFYTGFFNDFYNLDENLGTRISEEAVRNSWNTAASGGFFAAAAAPTTWYTDFRADIPRIDVPALILHGTGDRTLPIENTARVFHKALPSAEYVEVEGAPHGLLWTHAEEVNTALLAFLAKAQEAQKQKLLTEVETYVLSIIPSGPLKAEIAQRLEDVFAGGGRWGADCAWHLGELVWCTAGWEGGDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQLEHHHHHH

SEQ ID NO: 3 - protein, 470 aa, synthetic construct
MPFITVGQENSTSIDLYYEDHGTGTPVVLIHGFPLSGHSWERQSAALLDAGYRVITYDRRGFGQSSQPTTGYDYDTFAADLNTVLETLDLQDAVLVGFSMGTGEVARYVSSYGTARIAAVAFLASLEPFLLKTDDNPDGAAPQEFFDGIVAAVKADRYAFYTGFFNDFYNLDENLGTRISEEAVRNSWNTAASGGFFAAAAAPTTWYTDFRADIPRIDVPALILHGTGDRTLPIENTARVFHKALPSAEYVEVEGAPHGLLWTHAEEVNTALLAFLAKAQEAQKQKLLTEVETYVLSIIPSGPLKAEIAQRLEDVFAGGADCAWHLGELVWCTAGDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQLEHHHHHH